(19) United States
(12) Patent Application Publication
(10) Pub. No.: US 2026/0243746 A1
(43) Pub. Date: Aug. 20, 2026

(54) Automatic Defect Classification Analysis

(71) Applicant: KLA Corporation, Milpitas, CA (US)

(72) Inventor: Tingzhu Liu, Qingdao (CN)

(21) Appl. No.: 19/056,379

(22) Filed: Feb. 18, 2025

(51) Int. Cl. G01N 33/00; G01N 21/88; G01N 21/95; G01N 23/00; G06T 7/00; G06V 10/764

(52) U.S. Cl. CPC G01N 33/0095; G01N 21/8851; G01N 21/9501; G01N 23/00; G06T 7/0004; G06V 10/764; G01N 2021/8854; G06T 2207/30148; G06T 2207/30164

(57) ABSTRACT

Automatic defect classifications and manual defect classifications of a workpiece (such as a semiconductor wafer) are assembled along perpendicular axes of a matrix. Matches between corresponding entries of the automatic defect classifications and the manual defect classifications are determined. A purity rating, a contribution rating, and an accuracy rating are determined based on the matrix.

### FIELD OF THE DISCLOSURE

This disclosure relates to defect classification during semiconductor inspection.

### BACKGROUND OF THE DISCLOSURE

Evolution of the semiconductor manufacturing industry is placing greater demands on yield management and, in particular, on metrology and inspection systems. Critical dimensions continue to shrink, yet the industry needs to decrease time for achieving high-yield, high-value production. Minimizing the total time from detecting a yield problem to fixing it maximizes the return-on-investment for a semiconductor manufacturer.

Fabricating semiconductor devices, such as logic and memory devices, typically includes processing a semiconductor wafer using a large number of fabrication processes to form various features and multiple levels of the semiconductor devices. For example, lithography is a semiconductor fabrication process that involves transferring a pattern from a reticle to a photoresist arranged on a semiconductor wafer. Additional examples of semiconductor fabrication processes include, but are not limited to, chemical-mechanical polishing (CMP), etching, deposition, and ion implantation. An arrangement of multiple semiconductor devices fabricated on a single semiconductor wafer may be separated into individual semiconductor devices.

Inspection processes are used at various steps during semiconductor manufacturing to detect defects on wafers to promote higher yield in the manufacturing process and, thus, higher profits. Inspection has always been an important part of fabricating semiconductor devices such as integrated circuits (ICs). However, as the dimensions of semiconductor devices decrease, inspection becomes even more important to the successful manufacture of acceptable semiconductor devices because smaller defects can cause the devices to fail. For instance, as the dimensions of semiconductor devices decrease, detection of defects of decreasing size has become necessary because even relatively small defects may cause unwanted aberrations in the semiconductor devices.

As design rules shrink, however, semiconductor manufacturing processes may be operating closer to the limitation on the performance capability of the processes. In addition, smaller defects can have an impact on the electrical parameters of the device as the design rules shrink, which drives more sensitive inspections. As design rules shrink, the population of potentially yield-relevant defects detected by inspection grows dramatically, and the population of nuisance defects detected by inspection also increases dramatically. Therefore, more defects may be detected on the wafers, and correcting the processes to eliminate all of the defects may be difficult and expensive. Determining which of the defects actually have an effect on the electrical parameters of the devices and the yield may allow process control methods to be focused on those defects while largely ignoring others. Furthermore, at smaller design rules, process-induced failures, in some cases, tend to be systematic. That is, process-induced failures tend to fail at predetermined design patterns often repeated many times within the design. Elimination of spatially-systematic, electrically-relevant defects can have an impact on yield.

Classifying defects found on wafers and other workpieces has, therefore, become increasingly important to determine what kinds of defects are present on the wafers in addition to distinguishing defect types of interest from other defect types. Several fully automatic defect classification (ADC) tools are now available. Typically, these tools use classification “recipes” to perform defect classification. A “recipe” can be generally defined as a set of instructions that define an operation to be performed by a tool and that are provided to and run on the tool upon request by a user. Classification recipes are typically generated using previously acquired data for specific defect classes that may be assembled in a suitable database. In the simplest implementation, the ADC tool can then compare unknown defects to those included in the specific defect classes to determine which defect class the unknown defect is most like. More complicated algorithms can be used by the ADC tool to determine the defect class to which the unknown defect most likely belongs.

Sometimes ADC is performed after inspection of a wafer. However, some systems and methods have been developed that can be used to perform ADC during inspection or “on-the-fly.” In other words, the electrical signals produced by each of the detectors are processed separately to determine if each detector has detected a defect. At any time that a defect is detected in the electrical signals produced by one of the detectors, the electrical signals produced by all the detectors are analyzed collectively to determine scattered light attributes of the defect such as reflected light intensity, reflected light volume, reflected light linearity, and reflected light asymmetry. The defect is then classified (e.g., as a pattern defect or a particle defect) based on these attributes.

ADC performance must be evaluated to ensure accuracy. In the past, an ADC defect code was manually checked. A sample of these results was evaluated to determine the performance of the ADC model. However, this was extremely slow and labor intensive. It also did not capture all the ADC defect codes without significant time investment. Improved techniques and systems are needed.

### BRIEF SUMMARY OF THE DISCLOSURE

A method is provided in a first embodiment. The method includes receiving a plurality of automatic defect classifications for a workpiece and a plurality of manual defect classifications for the workpiece at a processor. The plurality of automatic defect classifications and the plurality of manual defect classifications are assembled along perpendicular axes of a matrix using the processor. Matches between corresponding entries of the plurality of automatic defect classifications and the plurality of manual defect classifications are determined with the processor. A purity rating, contribution rating, and accuracy rating are determined based on the matrix using the processor. The purity evaluates correctness of classification for defect types. The contribution rating evaluates a percentage of defects that were classified. The accuracy rating evaluates a percentage of defects that were classified correctly.

The method can include inspecting one or more workpieces with a semiconductor inspection system. The method also can include generating a file that includes the plurality of automatic defect classifications.

The workpiece may be a semiconductor wafer. The plurality of automatic defect classifications may be received in a text file.

The method can include updating the manual defect classifications using at least one of purity rating, the contribution rating, or the accuracy rating.

A system is provided in a second embodiment. The system includes a semiconductor inspection system with a particle beam source that generates a particle beam; a stage that supports a workpiece in a path of the particle beam; and a detector that receives the particle beam returned from the workpiece. A processor is in electronic communication with the detector. The processor is configured to receive a plurality of automatic defect classifications for the workpiece; receive a plurality of manual defect classifications for the workpiece; assemble the plurality of automatic defect classifications and the plurality of manual defect classifications along perpendicular axes of a matrix; determine matches between corresponding entries of the plurality of automatic defect classifications and the plurality of manual defect classifications; determine a purity rating based on the matrix; determine a contribution rating based on the matrix; and determine an accuracy rating based on the matrix. The purity evaluates correctness of classification for defect types. The contribution rating evaluates a percentage of defects that were classified. The accuracy rating evaluates a percentage of defects that were classified correctly.

The processor can be further configured to generate a file that includes the plurality of automatic defect classifications. The file may be a text file.

The workpiece may be a semiconductor wafer. The particle beam may be a beam of light or an electron beam.

A non-transitory computer-readable storage medium is provided in a third embodiment. The non-transitory computer-readable storage medium includes one or more programs for executing the following steps on one or more computing devices. The steps includes receiving a plurality of automatic defect classifications for a workpiece; receiving a plurality of manual defect classifications for the workpiece; assembling the plurality of automatic defect classifications and the plurality of manual defect classifications along perpendicular axes of a matrix; determining matches between corresponding entries of the plurality of automatic defect classifications and the plurality of manual defect classifications; determining a purity rating based on the matrix; determining a contribution rating based on the matrix; and determining an accuracy rating based on the matrix. The purity evaluates correctness of classification for defect types. The contribution rating evaluates a percentage of defects that were classified. The accuracy rating evaluates a percentage of defects that were classified correctly.

The workpiece may be a semiconductor wafer.

The plurality of automatic defect classifications may be received in a text file.

The steps can include sending instructions to a semiconductor inspection system to inspect the workpiece.

### DESCRIPTION OF THE DRAWINGS

For a fuller understanding of the nature and objects of the disclosure, reference should be made to the following detailed description taken in conjunction with the accompanying drawings, in which:

FIG. 1 is a flowchart of a method in accordance with the present disclosure;

FIG. 2 shows two exemplary matrices using the method of FIG. 1; and

FIG. 3 is an exemplary semiconductor inspection tool in accordance with the present disclosure.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, process step, and electronic changes may be made without departing from the scope of the disclosure. Accordingly, the scope of the disclosure is defined only by reference to the appended claims.

In semiconductor manufacturing, ADC systems are used to group defects formed during manufacturing processes into different defect bins. The defects can then be studied, which can provide process optimization and yield improvement. Defect classification by ADC systems are usually not 100% accurate. Embodiments disclosed herein can be used to determine the performance of the ADC system, such as for a batch of wafers or other workpieces or all the defects classified on a wafer or other workpiece. Classification accuracy, purity, and contribution can be determined, which can help monitor the ADC system’s performance.

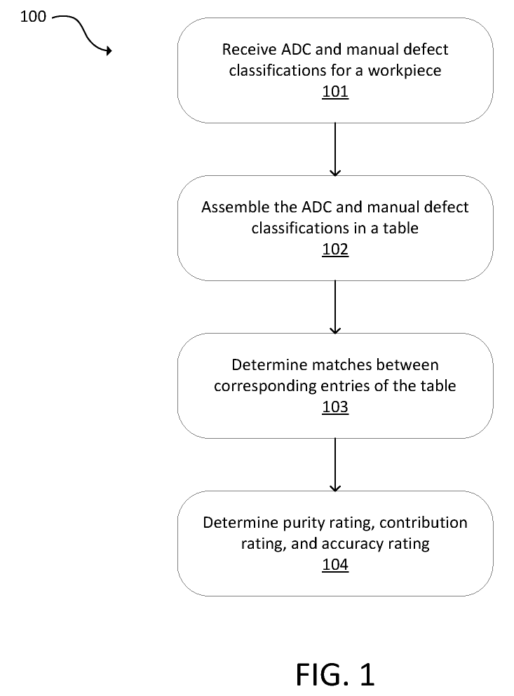

FIG. 1 is a flowchart of a method 100. The steps of the method 100 can use a processor. At 101, automatic defect classifications (ADC) and manual defect classifications for a workpiece are received. The workpiece may be, for example, a semiconductor wafer. The automatic defect classifications can be received in the form of a text file or other data reporting file, such as a KLARF file used by KLA Corporation. The file with the automatic defect classifications can include a code or description of the defect. The file also may include the defect’s location on the workpiece and general information about the workpiece.

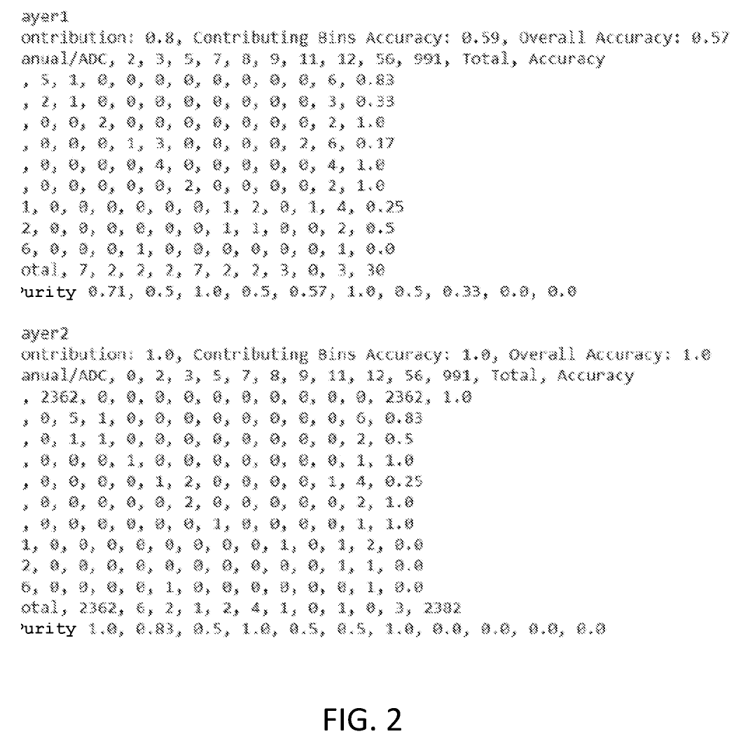

A KLARF file is text file that contains multiple columns and rows. Each row represents one defect. The value for each column describes an aspect of the defect. For example, the first column can the defect ID number and the second and third column can be the x, y coordinate that describes the location of the defect. The defect code given by an ADC system is recorded in one of the columns. Similarly, the manual code is also recorded in the KLARF file. Examples of a text file are shown in FIG. 2.

The automatic defect classifications can be generated using an ADC system. An inspection tool, such as a semiconductor inspection tool, can image the workpiece and identify defects. In an instance, a semiconductor inspection system inspects one or more workpieces to generate the results. For example, the semiconductor inspection system can generate one or more images of the workpiece that are evaluated during inspection to determine defects. These inspection results are used for later analysis using the embodiments disclosed herein. The type of defect can be added to the text file or other data reporting file.

In an embodiment, the manual defect classifications can be defect classifications associated with the sample images. For example, the manual defect classifications can be a series of defect codes that represent different types of defects. In another embodiment, the manual defect classifications can be classifications of the same defects as the automatic defect classifications reviewed by a technician. For manual classification, a technician reviews the defect image to decide the defect type, and then assigns a corresponding defect code. For example, code 1 can be used for particle defects. Different semiconductor manufacturers have different defect code definitions. The manual defect codes can be exported together with ADC codes in a KLARF file or another type of file.

Turning back to FIG. 1, the automatic defect classifications and manual defect classifications are assembled in a matrix at 102. The automatic defect classifications can be projected along one axis, and the manual defect classifications can be projected along the perpendicular axis. The ADC codes and manual codes in a KLARF file are stored in different columns. The Python scripts reads and extracts the information from the KLARF file and organizes them in an output format such that the automatic defect classifications and manual defect classifications are arranged on perpendicular axes of a matrix.

Two examples are shown in FIG. 2., with the automatic defect classifications across the horizontal axis in the top row and the manual defect classifications along the left-most vertical axis. The matrix can be part of a text file or other file format. The rows are manual codes, while the columns are ADC codes. The data inside is the number of defects. In the first row of FIG. 2, 6 (5+1) defects were manually classified as code 2 defects. Five of these were classified by ADC as code 2. One was misclassified by ADC as code 3. This resulted in a determination of 0.83 for the row.

In an instance, a Python script is used to assemble the matrix. The output format for the matrix may be a .txt file. The script reads and extracts defects ADC and manual codes from a KLARF file or another type of file and then computes the performance data for the ADC system in terms of purity, accuracy, and contribution. The data forms the basis to judge how the ADC system performs and what should be done to make it perform better. While described as a Python script, other programming languages can be used to perform this function. The function described herein reduces the processing power requirements. Previous techniques that automatically calculated ADC performance were part of a bigger system, which required a dedicated server. The script disclosed herein can be run on any computer with Python or another software program installed, which can drastically reduce the necessary processing power.

Turning back to FIG. 1, matches between corresponding entries of the automatic defect classifications and manual defect classifications on the matrix are determined at 103. The script determines if these entries are the same.

At 104, a purity rating, contribution rating, and accuracy rating are determined based on the matrix. A purity rating evaluates the correctness of classification for defect types. A purity rating can be defined as the number of correctly classified defects in an ADC bin divided by the total number of defects in that particular ADC bin. A contribution rating evaluates a percentage of defects that were classified. A contribution rating can be defined as the total number of defects in ADC bins that meet the purity threshold divided by the total number of defects. An accuracy rating evaluates a percentage of defects that were classified correctly. An accuracy rating can be defined as the number of defects of a given class code correctly classified by ADC divided by the total number of defects for that given class code.

The results of the method 100 can be used for process control. The results may be used to judge how their ADC system is performing, such as determining where the ADC system is doing well and where it is not. Actions can then be taken to improve ADC system performance. For example, classification methodology can be adjusted. The ADC may be retrained or sensitivity may be adjusted for one or more defect types to improve performance. A good performing ADC system can reduce manual labor to classify defects. The correctly classified defects data can be the basis for process control and yield improvement. This adjustment of classification methodology may be manual or automatic. In an instance, the purity rating, contribution rating, and/or accuracy rating for one or more defect types can be used to automatically adjust the ADC process.

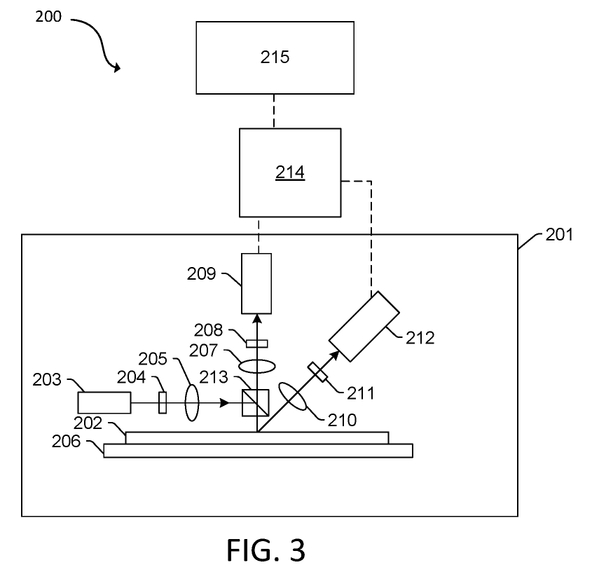

One embodiment of a semiconductor inspection system 200 is shown in FIG. 3. The semiconductor inspection system 200 includes optical based subsystem 201. In general, the optical based subsystem 201 is configured for generating optical based output for a workpiece 202 by directing light to (or scanning light over) and detecting light from the workpiece 202. In one embodiment, the workpiece 202 includes a semiconductor wafer. The wafer may include any wafer known in the art. In another embodiment, the workpiece 202 includes a reticle. The reticle may include any reticle known in the art.

In the embodiment of the semiconductor inspection system 200 shown in FIG. 3, optical based subsystem 201 includes an illumination subsystem configured to direct light to workpiece 202. The illumination subsystem includes at least one light source. For example, as shown in FIG. 3, the illumination subsystem includes light source 203. In one embodiment, the illumination subsystem is configured to direct the light to the workpiece 202 at one or more angles of incidence, which may include one or more oblique angles and/or one or more normal angles. For example, as shown in FIG. 3, light from light source 203 is directed through optical element 204 and then lens 205 to workpiece 202 at an oblique angle of incidence. The oblique angle of incidence may include any suitable oblique angle of incidence, which may vary depending on, for instance, characteristics of the workpiece 202.

The optical based subsystem 201 may be configured to direct the light to the workpiece 202 at different angles of incidence at different times. For example, the optical based subsystem 201 may be configured to alter one or more characteristics of one or more elements of the illumination subsystem such that the light can be directed to the workpiece 202 at an angle of incidence that is different than that shown in FIG. 3. In one such example, the optical based subsystem 201 may be configured to move light source 203, optical element 204, and lens 205 such that the light is directed to the workpiece 202 at a different oblique angle of incidence or a normal (or near normal) angle of incidence.

In some instances, the optical based subsystem 201 may be configured to direct light to the workpiece 202 at more than one angle of incidence at the same time. For example, the illumination subsystem may include more than one illumination channel, one of the illumination channels may include light source 203, optical element 204, and lens 205 as shown in FIG. 3 and another of the illumination channels (not shown) may include similar elements, which may be configured differently or the same, or may include at least a light source and possibly one or more other components such as those described further herein. If such light is directed to the workpiece 202 at the same time as the other light, one or more characteristics (e.g., wavelength, polarization, etc.) of the light directed to the workpiece 202 at different angles of incidence may be different such that light resulting from illumination of the workpiece 202 at the different angles of incidence can be discriminated from each other at the detector(s).

In another instance, the illumination subsystem may include only one light source (e.g., light source 203 shown in FIG. 3) and light from the light source may be separated into different optical paths (e.g., based on wavelength, polarization, etc.) by one or more optical elements (not shown) of the illumination subsystem. Light in each of the different optical paths may then be directed to the workpiece 202. Multiple illumination channels may be configured to direct light to the workpiece 202 at the same time or at different times (e.g., when different illumination channels are used to sequentially illuminate the specimen). In another instance, the same illumination channel may be configured to direct light to the workpiece 202 with different characteristics at different times. For example, in some instances, optical element 204 may be configured as a spectral filter and the properties of the spectral filter can be changed in a variety of different ways (e.g., by swapping out the spectral filter) such that different wavelengths of light can be directed to the workpiece 202 at different times. The illumination subsystem may have any other suitable configuration known in the art for directing the light having different or the same characteristics to the workpiece 202 at different or the same angles of incidence sequentially or simultaneously.

In one embodiment, light source 203 may include a broadband plasma (BBP) source. In this manner, the light generated by the light source 203 and directed to the workpiece 202 may include broadband light. However, the light source may include any other suitable light source such as a laser. The laser may include any suitable laser known in the art and may be configured to generate light at any suitable wavelength or wavelengths known in the art. In addition, the laser may be configured to generate light that is monochromatic or nearly monochromatic. In this manner, the laser may be a narrowband laser. The light source 203 may also include a polychromatic light source that generates light at multiple discrete wavelengths or wavebands.

Light from optical element 204 may be focused onto workpiece 202 by lens 205. Although lens 205 is shown in FIG. 3 as a single refractive optical element, it is to be understood that, in practice, lens 205 may include a number of refractive and/or reflective optical elements that in combination focus the light from the optical element to the specimen. The illumination subsystem shown in FIG. 3 and described herein may include any other suitable optical elements (not shown). Examples of such optical elements include, but are not limited to, polarizing component(s), spectral filter(s), spatial filter(s), reflective optical element(s), apodizer(s), beam splitter(s) (such as beam splitter 213), aperture(s), and the like, which may include any such suitable optical elements known in the art. In addition, the optical based subsystem 201 may be configured to alter one or more of the elements of the illumination subsystem based on the type of illumination to be used for generating the optical based output.

The optical based subsystem 201 may also include a scanning subsystem configured to cause the light to be scanned over the workpiece 202. For example, the optical based subsystem 201 may include stage 206 on which workpiece 202 is disposed during optical based output generation. The scanning subsystem may include any suitable mechanical and/or robotic assembly (that includes stage 206) that can be configured to move the workpiece 202 such that the light can be scanned over the workpiece 202. In addition, or alternatively, the optical based subsystem 201 may be configured such that one or more optical elements of the optical based subsystem 201 perform scanning of the light over the workpiece 202. The light may be scanned over the workpiece 202 in any suitable fashion such as in a serpentine-like path or in a spiral path.

The optical based subsystem 201 further includes one or more detection channels. At least one of the one or more detection channels includes a detector configured to detect light from the workpiece 202 due to illumination of the workpiece 202 by the subsystem and to generate output responsive to the detected light. For example, the optical based subsystem 201 shown in FIG. 3 includes two detection channels, one formed by collector 207, element 208, and detector 209 and another formed by collector 210, element 211, and detector 212. As shown in FIG. 3, the two detection channels are configured to collect and detect light at different angles of collection. In some instances, both detection channels are configured to detect scattered light, and the detection channels are configured to detect light that is scattered at different angles from the workpiece 202. However, one or more of the detection channels may be configured to detect another type of light from the workpiece 202 (e.g., reflected light).

As further shown in FIG. 3, both detection channels are shown positioned in the plane of the paper and the illumination subsystem is also shown positioned in the plane of the paper. Therefore, in this embodiment, both detection channels are positioned in (e.g., centered in) the plane of incidence. However, one or more of the detection channels may be positioned out of the plane of incidence. For example, the detection channel formed by collector 210, element 211, and detector 212 may be configured to collect and detect light that is scattered out of the plane of incidence. Therefore, such a detection channel may be commonly referred to as a “side” channel, and such a side channel may be centered in a plane that is substantially perpendicular to the plane of incidence.

Although FIG. 3 shows an embodiment of the optical based subsystem 201 that includes two detection channels, the optical based subsystem 201 may include a different number of detection channels (e.g., only one detection channel or two or more detection channels). In one such instance, the detection channel formed by collector 210, element 211, and detector 212 may form one side channel as described above, and the optical based subsystem 201 may include an additional detection channel (not shown) formed as another side channel that is positioned on the opposite side of the plane of incidence. Therefore, the optical based subsystem 201 may include the detection channel that includes collector 207, element 208, and detector 209 and that is centered in the plane of incidence and configured to collect and detect light at scattering angle(s) that are at or close to normal to the workpiece 202 surface. This detection channel may therefore be commonly referred to as a “top” channel, and the optical based subsystem 201 may also include two or more side channels configured as described above. As such, the optical based subsystem 201 may include at least three channels (i.e., one top channel and two side channels), and each of the at least three channels has its own collector, each of which is configured to collect light at different scattering angles than each of the other collectors.

As described further above, each of the detection channels included in the optical based subsystem 201 may be configured to detect scattered light. Therefore, the optical based subsystem 201 shown in FIG. 3 may be configured for dark field (DF) output generation for workpieces 202. However, the optical based subsystem 201 may also or alternatively include detection channel(s) that are configured for bright field (BF) output generation for workpieces 202. In other words, the optical based subsystem 201 may include at least one detection channel that is configured to detect light specularly reflected from the workpiece 202. Therefore, the optical based subsystems 201 described herein may be configured for only DF, only BF, or both DF and BF imaging. Although each of the collectors are shown in FIG. 3 as single refractive optical elements, it is to be understood that each of the collectors may include one or more refractive optical die(s) and/or one or more reflective optical element(s).

The one or more detection channels may include any suitable detectors known in the art. For example, the detectors may include photo-multiplier tubes (PMTs), charge coupled devices (CCDs), time delay integration (TDI) cameras, and any other suitable detectors known in the art. The detectors may also include non-imaging detectors or imaging detectors. In this manner, if the detectors are non-imaging detectors, each of the detectors may be configured to detect certain characteristics of the scattered light such as intensity but may not be configured to detect such characteristics as a function of position within the imaging plane. As such, the output that is generated by each of the detectors included in each of the detection channels of the optical based subsystem may be signals or data, but not image signals or image data. In such instances, a processor such as processor 214 may be configured to generate images of the workpiece 202 from the non-imaging output of the detectors. However, in other instances, the detectors may be configured as imaging detectors that are configured to generate imaging signals or image data. Therefore, the optical based subsystem may be configured to generate optical images or other optical based output described herein in several ways.

It is noted that FIG. 3 is provided herein to generally illustrate a configuration of an optical based subsystem 201 that may be included in the system embodiments described herein or that may generate optical based output that is used by the system embodiments described herein. The optical based subsystem 201 configuration described herein may be altered to optimize the performance of the optical based subsystem 201 as is normally performed when designing a commercial output acquisition system. In addition, the systems described herein may be implemented using an existing system (e.g., by adding functionality described herein to an existing system). For some such systems, the methods described herein may be provided as optional functionality of the system (e.g., in addition to other functionality of the system). Alternatively, the system described herein may be designed as a completely new system.

The processor 214 may be coupled to the components of the semiconductor inspection system 200 in any suitable manner (e.g., via one or more transmission media, which may include wired and/or wireless transmission media) such that the processor 214 can receive output. The processor 214 may be configured to perform several functions using the output. The semiconductor inspection system 200 can receive instructions or other information from the processor 214. The processor 214 and/or the electronic data storage unit 215 optionally may be in electronic communication with a wafer inspection tool, a wafer metrology tool, or a wafer review tool (not illustrated) to receive additional information or send instructions. For example, the processor 214 and/or the electronic data storage unit 215 can be in electronic communication with a scanning electron microscope.

The processor 214, other system(s), or other subsystem(s) described herein may be part of various systems, including a personal computer system, image computer, mainframe computer system, workstation, network appliance, internet appliance, or other device. The subsystem(s) or system(s) may also include any suitable processor known in the art, such as a parallel processor. In addition, the subsystem(s) or system(s) may include a platform with high-speed processing and software, either as a standalone or a networked tool.

The processor 214 and electronic data storage unit 215 may be disposed in or otherwise part of the semiconductor inspection system 200 or another device. In an example, the processor 214 and electronic data storage unit 215 may be part of a standalone control unit or in a centralized quality control unit. Multiple processors 214 or electronic data storage units 215 may be used.

The processor 214 may be implemented in practice by any combination of hardware, software, and firmware. Also, its functions as described herein may be performed by one unit, or divided up among different components, each of which may be implemented in turn by any combination of hardware, software and firmware. Program code or instructions for the processor 214 to implement various methods and functions may be stored in readable storage media, such as a memory in the electronic data storage unit 215 or other memory.

If the semiconductor inspection system 200 includes more than one processor 214, then the different subsystems may be coupled to each other such that images, data, information, instructions, etc. can be sent between the subsystems. For example, one subsystem may be coupled to additional subsystem(s) by any suitable transmission media, which may include any suitable wired and/or wireless transmission media known in the art. Two or more of such subsystems may also be effectively coupled by a shared computer-readable storage medium (not shown).

The processor 214 may be configured to perform several functions using the output of the semiconductor inspection system 200 or other output. For instance, the processor 214 may be configured to send the output to an electronic data storage unit 215 or another storage medium. The processor 214 may be configured according to any of the embodiments described herein. The processor 214 also may be configured to perform other functions or additional steps using the output of the system 200 or using images or data from other sources.

Various steps, functions, and/or operations of semiconductor inspection system 200 and the methods disclosed herein are carried out by one or more of the following: electronic circuits, logic gates, multiplexers, programmable logic devices, ASICs, analog or digital controls/switches, microcontrollers, or computing systems. Program instructions implementing methods such as those described herein may be transmitted over or stored on carrier medium. The carrier medium may include a storage medium such as a read-only memory, a random-access memory, a magnetic or optical disk, a non-volatile memory, a solid-state memory, a magnetic tape, and the like. A carrier medium may include a transmission medium such as a wire, cable, or wireless transmission link. For instance, the various steps described throughout the present disclosure may be carried out by a single processor 214 or, alternatively, multiple processors 214. Moreover, different sub-systems of the semiconductor inspection system 200 may include one or more computing or logic systems. Therefore, the above description should not be interpreted as a limitation on the present disclosure but merely an illustration.

In an instance, the processor 214 is in communication with the semiconductor inspection system 200. The processor 214 is configured to perform, for example, the method 100. Automatic defect classifications and manual defect classifications are assembled along perpendicular axes of a matrix. Matches between corresponding entries of the plurality of automatic defect classifications and the plurality of manual defect classifications can be determined. A purity rating, contribution rating, and accuracy rating can then be determined.

An additional embodiment relates to a non-transitory computer-readable medium storing program instructions executable on a controller for performing a computer-implemented method, as disclosed herein. In particular, as shown in FIG. 3, electronic data storage unit 215 or other storage medium may contain non-transitory computer-readable medium that includes program instructions executable on the processor 214. The computer-implemented method may include any step(s) of any method(s) described herein, including method 100.

The program instructions may be implemented in any of various ways, including procedure-based techniques, component-based techniques, and/or object-oriented techniques, among others. For example, the program instructions may be implemented using Python, ActiveX controls, C++ objects, JavaBeans, Microsoft Foundation Classes (MFC), Streaming SIMD Extension (SSE), or other technologies or methodologies, as desired.

While the semiconductor inspection system is described as using an optical beam, other particle beams can be used. For example, the semiconductor inspection system can be an electron beam system that directs the electron beam toward a workpiece. The semiconductor inspection system also can use ions directed toward the workpiece.

Each of the steps of the method may be performed as described herein. The methods also may include any other step(s) that can be performed by the processor and/or computer subsystem(s) or system(s) described herein. The steps can be performed by one or more computer systems, which may be configured according to any of the embodiments described herein. In addition, the methods described above may be performed by any of the system embodiments described herein.

Although the present disclosure has been described with respect to one or more particular embodiments, it will be understood that other embodiments of the present disclosure may be made without departing from the scope of the present disclosure. Hence, the present disclosure is deemed limited only by the appended claims and the reasonable interpretation thereof.

What is claimed is:

1. A method comprising:
receiving, at a processor, a plurality of automatic defect classifications for a workpiece;
receiving, at the processor, a plurality of manual defect classifications for the workpiece;
assembling, using the processor, the plurality of automatic defect classifications and the plurality of manual defect classifications along perpendicular axes of a matrix;
determining, using the processor, matches between corresponding entries of the plurality of automatic defect classifications and the plurality of manual defect classifications;
determining, using the processor, a purity rating based on the matrix, wherein the purity rating evaluates correctness of classification for defect types;
determining, using the processor, a contribution rating based on the matrix, wherein the contribution rating evaluates a percentage of defects that were classified; and
determining, using the processor, an accuracy rating based on the matrix, wherein the accuracy rating evaluates a percentage of defects that were classified correctly.

2. The method of claim 1, further comprising inspecting one or more workpieces with a semiconductor inspection system.

3. The method of claim 2, further comprising generating a file that includes the plurality of automatic defect classifications.

4. The method of claim 1, wherein the workpiece is a semiconductor wafer.

5. The method of claim 1, wherein the plurality of automatic defect classifications are received in a text file.

6. The method of claim 1, further comprising updating the manual defect classifications using at least one of purity rating, the contribution rating, or the accuracy rating.

7. A system comprising:
a semiconductor inspection system that includes:
a particle beam source that generates a particle beam;
a stage that supports a workpiece in a path of the particle beam; and
a detector that receives the particle beam returned from the workpiece; and
a processor in electronic communication with the detector, wherein the processor is configured to:
receive a plurality of automatic defect classifications for the workpiece;
receive a plurality of manual defect classifications for the workpiece;
assemble the plurality of automatic defect classifications and the plurality of manual defect classifications along perpendicular axes of a matrix;
determine matches between corresponding entries of the plurality of automatic defect classifications and the plurality of manual defect classifications;
determine a purity rating based on the matrix, wherein the purity rating evaluates correctness of classification for defect types;
determine a contribution rating based on the matrix, wherein the contribution rating evaluates a percentage of defects that were classified; and
determine an accuracy rating based on the matrix, wherein the accuracy rating evaluates a percentage of defects that were classified correctly.

8. The system of claim 7, wherein the processor is further configured to generate a file that includes the plurality of automatic defect classifications.

9. The system of claim 8, wherein the file is a text file.

10. The system of claim 7, wherein the workpiece is a semiconductor wafer.

11. The system of claim 7, wherein the particle beam is a beam of light.

12. The system of claim 7, wherein the particle beam is an electron beam.

13. A non-transitory computer-readable storage medium, comprising one or more programs for executing the following steps on one or more computing devices:
receiving a plurality of automatic defect classifications for a workpiece;
receiving a plurality of manual defect classifications for the workpiece;
assembling the plurality of automatic defect classifications and the plurality of manual defect classifications along perpendicular axes of a matrix;
determining matches between corresponding entries of the plurality of automatic defect classifications and the plurality of manual defect classifications;
determining a purity rating based on the matrix, wherein the purity rating evaluates correctness of classification for defect types;
determining a contribution rating based on the matrix, wherein the contribution rating evaluates a percentage of defects that were classified; and
determining an accuracy rating based on the matrix, wherein the accuracy rating evaluates a percentage of defects that were classified correctly.

14. The non-transitory computer-readable storage medium of claim 13, wherein the workpiece is a semiconductor wafer.

15. The non-transitory computer-readable storage medium of claim 13, wherein the plurality of automatic defect classifications are received in a text file.

16. The non-transitory computer-readable storage medium of claim 13, wherein the steps further comprising sending instructions to a semiconductor inspection system to inspect the workpiece.
